(19) United States
(12) Patent Application Publication
(10) Pub. No.: US 2025/0304947 A1
(43) Pub. Date: Oct. 2, 2025

(54) COMPOSITIONS AND METHODS FOR NUCLEIC ACID EXTRACTION AND PURIFICATION

(71) Applicant: OXFORD NANOPORE TECHNOLOGIES PLC., Oxford (GB)

(72) Inventor: Simon Charles Mayes, Oxford (GB)

(73) Assignee: OXFORD NANOPORE TECHNOLOGIES PLC., Oxford (GB)

(21) Appl. No.: 18/866,105

(22) PCT Filed: May 17, 2023
(86) PCT No.: PCT/GB2023/051303

§ 371 Date: Nov. 15, 2024

Related U.S. Application Data
(60) Provisional application No. 63/343,409, filed on May 18, 2022.

(51) Int. Cl. C12N 15/10

(52) U.S. Cl. CPC C12N 15/1006

(57) ABSTRACT

Aspects of the disclosure relate to methods for extracting and/or purifying nucleic acids from biological samples. The disclosure is based, in part, on methods comprising contacting DNA in a sample with a polyhedral, rigid substrate under conditions under which the nucleic acids interact (e.g., adsorb or bind) with the substrate to form aggregates, and eluting isolated or purified DNA from the substrate after washing or other sample processing techniques. In some embodiments, methods described by the disclosure result in less sheared isolated or purified DNA relative to previously employed substrates. The resulting isolated nucleic acids may be used for sequencing, for example ultra-long read DNA sequencing.

### RELATED APPLICATIONS

This application is a national stage filing under 35 U.S.C. § 371 of international PCT application PCT/GB2023/051303, filed May 17, 2023, which claims priority under 35 U.S.C. § 119(e) to U.S. provisional patent application, U.S. Ser. No. 63/343,409, filed May 18, 2022, the entire contents of each of which are herein incorporated by reference.

### BACKGROUND

Many techniques for analyzing nucleic acids, for example characterization of nucleic acids by sequencing, are heavily dependent upon the quality of nucleic acids used as input for the analysis. Sample preparation typically includes the steps of tissue lysis, nucleic acid extraction, and purification.

### SUMMARY

Aspects of the disclosure relate to compositions and methods for extracting and/or purifying nucleic acids from a biological sample. The disclosure is based, in part, on polyhedral, rigid substrates that, when contacted with nucleic acids of a biological sample, interact (e.g., adsorb, bind, chelate etc.) with large DNA molecules in the sample. The large DNA molecules interacting with the substrate can then be isolated from the biological sample with less damage (e.g., DNA shearing) than previously utilized DNA extraction or purification methods.

Accordingly, in some aspects, the disclosure provides a method for isolating nucleic acids from a biological sample, the method comprising: obtaining a biological sample comprising nucleic acids; contacting the biological sample with a polyhedral, rigid substrate; adsorbing the nucleic acids of the sample to the polyhedral, rigid substrate; washing the polyhedral, rigid substrate to remove non-nucleic acid components of the biological sample; releasing the nucleic acids from the polyhedral, rigid substrate to produce isolated nucleic acids from the biological sample.

In some embodiments, nucleic acids comprise DNA. In some embodiments, DNA is genomic DNA (gDNA). In some embodiments, nucleic acids comprise high molecular weight (HMW) DNA. In some embodiments, HMW DNA comprises DNA of at least 30 kb in size. In some embodiments, HMW DNA comprises DNA of at least 50 kb, at least 100 kb, at least 200 kb, at least 300 kb, at least 500 kb, or at least 1 Mb in size.

In some embodiments, a biological sample comprises a tissue sample, blood sample, tissue lysate, or cell lysate. In some embodiments, a tissue lysate or cell lysate has been previously obtained from a biological sample.

In some embodiments, obtaining a biological sample comprises lysing cells or tissue of the biological sample to produce a cell lysate or tissue lysate.

In some embodiments, a polyhedral, rigid substrate comprises or consists of between 4 and 50 faces. In some embodiments, a polyhedral, rigid substrate comprises or consists of 12 faces. In some embodiments, a polyhedral, rigid substrate forms a regular shape. In some embodiments, a polyhedral, rigid substrate forms an irregular shape.

In some embodiments, a polyhedral, rigid substrate forms a star shape. In some embodiments, a star shape is a regular star shape. In some embodiments, a star shape comprises five points. In some embodiments, each outer angle of a star shape is between 71 and 73 degrees (e.g., 71 degrees, 72 degrees, 73 degrees, or any angle therebetween). In some embodiments, each inner angle of each point of a star shape is between 53 and 56 degrees (e.g., 53 degrees, 54 degrees, 55 degrees, 56 degrees, or any angle therebetween, such as 54.3 degrees).

In some embodiments, each edge of a polyhedral, rigid substrate is substantially smooth. In some embodiments, each face of a polyhedral, rigid substrate is substantially smooth.

In some embodiments, a polyhedral, rigid substrate comprises an outer diameter between about 5 mm and about 7 mm in length. In some embodiments, a polyhedral, rigid substrate comprises an outer diameter of 6 mm in length. In some embodiments, a polyhedral, rigid substrate comprises a thickness between 0.1 mm and 0.5 mm.

In some embodiments, a polyhedral, rigid substrate comprises or consists of metal. In some embodiments, a metal comprises or consists of stainless steel. In some embodiments, stainless steel is 304 stainless steel, 316 stainless steel, 420 stainless steel, or 440 stainless steel.

In some embodiments, adsorbing a nucleic acids of the sample to the polyhedral, rigid substrate comprises providing conditions under which the nucleic acids of the sample nucleate on the surface of the substrate and form one or more nucleic acid aggregates. In some embodiments, the conditions comprise contacting the biological sample with isopropanol, spermine, water, or TE buffer.

In some embodiments, washing comprises centrifuging the biological sample after contacting the biological sample with a wash buffer. In some embodiments, washing comprises performing a wash step (e.g., contacting with wash buffer, centrifuging, removing wash buffer, etc.) more than one time (e.g., 2, 3, 4, 5, or more times).

In some embodiments, non-nucleic acid components removed from the biological sample comprise one or more proteins, RNA molecules, salts, carbohydrates, or other cellular debris.

In some embodiments, releasing the nucleic acids from the substrate comprises contacting the substrate with isopropanol, spermine, water, or TE buffer.

In some embodiments, isolated nucleic acids comprise or consist of genomic DNA (gDNA). In some embodiments, isolated nucleic acids comprise or consist of high molecular weight (HMW) DNA.

In some embodiments, isolated HMW DNA comprises at least 30 kb. In some embodiments, isolated nucleic acids comprise less sheared DNA than nucleic acid preparations prepared according to methods that utilize circular-shaped solid substrates or beads.

In some aspects, the disclosure provides a kit comprising (a) a microcentrifuge tube; and (b) a polyhedral, rigid substrate as described herein.

In some embodiments, the kit further comprises one or more buffers (e.g., lysis buffer, wash buffer, elution buffer, etc.). In some embodiments, the one or more buffers comprise spermine.

In some embodiments, the disclosure provides a method of nucleic acid (e.g., DNA) sequencing comprising: obtaining isolated nucleic acids according to a method as described herein, and sequencing the isolated nucleic acids using a sequencing apparatus (e.g., a sequencing apparatus suitable for ultra-long read sequencing).

### BRIEF DESCRIPTION OF DRAWINGS

FIG. 1 is a schematic depicting one embodiment of a polyhedral, rigid substrate as described by the disclosure. The substrate comprises a regular star-shaped, stainless steel substrate that binds nucleic acids (e.g., DNA) present in biological samples (e.g., cell or tissue lysates). All values shown in FIG. 1 are expressed in millimeters (mm), unless otherwise specified.

FIG. 2 shows representative data indicating that as the gDNA in a sample precipitates, it nucleates around the polyhedral rigid substrate (e.g., the metal star) and aggregates at the surface of the substrate.

FIG. 3 shows a photograph indicating constriction points formed by circular substrates. Such constriction points have been observed to contribute to shearing of DNA (e.g., gDNA), resulting in shorter DNA fragments being isolated from a biological sample.

FIG. 4 shows representative data indicating read length N50s from sequencing runs where a DNA library DNA was cleaned using rigid, polyhedral substrates described herein (e.g., metal star shapes).

### DETAILED DESCRIPTION

Aspects of the disclosure relate to compositions and methods for extracting and/or purifying nucleic acids from a biological sample. The disclosure is based, in part, on polyhedral, rigid substrates that, when contacted with nucleic acids of a biological sample, interact (e.g., adsorb, bind, chelate etc.) with the nucleic acids such that the nucleic acids can be isolated from the biological sample with less damage (e.g., DNA shearing) than previously utilized DNA extraction or purification methods.

Polyhedral Rigid Substrates Sequencing very long reads of DNA requires isolating large DNA molecules (e.g., high molecular weight (HMW) DNA) and preserving those DNA molecules through library preparation techniques. One challenge faced during the preservation of long DNA molecules is shearing of the DNA during DNA extraction or DNA purification procedures. For example, vigorous pipetting, pipetting through a narrow bore (e.g., non “wide bore”) pipette tip, and the use of certain substrates such as borosilicate beads all contribute to mechanical shearing of DNA. This shearing of template DNA results in reduced yields of HMW DNA molecules, thus lowering the integrity of long-read sequencing information obtained from such DNA.

The inventors have recognized and appreciated that nucleic acids (e.g., DNA) extracted or purified using substrates having rigid construction and certain non-circular geometries (e.g., polyhedral shapes, such as star shapes) is less damaged (e.g., has reduced shearing and/or results in obtaining longer DNA template molecules) than DNA that is extracted or purified using techniques employing beads, or certain circular substrates (e.g., as disclosed in International Patent Publication Number WO 2015/020818).

The polyhedral, rigid substrates described by the disclosure also provide improved DNA extraction or purification relative to previously-described flexible, polyhedral substrates. For example, the rigid substrates described herein allow for recovery of semi-eluted DNA because the rigid substrates are not spun to the bottom of a microcentrifuge tube during centrifugation, as happens with substrates made from less rigid materials.

As used herein, a “polyhedral” substrate refers to a three-dimensional substrate that comprises four or more faces. Examples of polyhedral substrates include but are not limited to three-dimensional substrates having a prism shape, pyramid shape, cube shape, tetrahedron shape, pentahedron shape, hexahedron shape, heptahedron shape, octahedron shape, nonahedron shape, decahedron shape, dodecahedron shape, icosahedron shape, etc. In some embodiments, a polyhedral substrate comprises between 4 and 50 (e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) faces. In some embodiments, a polyhedral substrate comprises between 6 and 15 faces. In some embodiments, a polyhedral substrate comprises 12 faces.

A polyhedron may be a regular polyhedron (e.g., a polyhedron that is highly symmetrical, edge-transitive, vertex-transitive and face-transitive) or an irregular polyhedron (e.g., formed by polygons having different shapes where all the elements are not the same).

In some embodiments, a polyhedron comprises a star polyhedron shape. A star polyhedron shape refers to a self-intersecting, uniform polyhedron that comprises star polygon faces and/or star polygon vertex figures. Examples of star polyhedron shapes include but are not limited to small stellated dodecahedrons, great icosahedrons, pentagrammic prisms, pentagrammic dipyramids, and star polytopes.

The number of points (also referred to as vertices) of a star polyhedron may vary. In some embodiments, a star polyhedron comprises or consists of between 5 and 92 vertices. In some embodiments, a star polyhedron comprises or consists of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, or 92 points.

A star polyhedron comprising points (e.g., vertices) has outer angles and inner angles. As used herein, an outer angle refers to the angle between one point (e.g., end of the point or vertex) of a star and an adjacent point of the star. An inner angle refers to the angle between the two sides of the angle forming a point of the star. The size of the angles forming inner or outer angles of a star polyhedron may vary. In some embodiments, the outer angle between points of a star polyhedron ranges from about 71 to 73 degrees (e.g., 71 degrees, 71.5 degrees, 72 degrees, 72.5 degrees, 73 degrees, or any angle therebetween). In some embodiments, each inner angle of each point of a star shape is between 53 and 56 degrees (e.g., 53 degrees, 54 degrees, 55 degrees, 56 degrees, or any angle therebetween, such as 54.3 degrees).

The disclosure is based, in part, on polyhedral, rigid substrates that are configured to fit within containers typically used for DNA extraction and/or purification without forming constriction points in the container. Examples of such containers include but are not limited to microcentrifuge tubes (e.g., Eppendorf tubes, etc.), test tubes, and conical vials. In some embodiments, the microcentrifuge tube is a 2.0 mL microcentrifuge tube, a 1.5 mL microcentrifuge tube, a 0.5 mL microcentrifuge tube, or a 0.2 mL microcentrifuge tube. In some embodiments, the microcentrifuge tube is a 1.5 mL microcentrifuge tube.

As used herein, “constriction points” refers to spaces between points of contact between a container wall and a solid substrate that form passages through which nucleic acids (e.g., DNA) travel during DNA extraction or purification procedures. Without wishing to be bound by any particular theory, the presence of constriction points in containers during DNA extraction or purification result in increased shearing of DNA during such procedures. In some embodiments, a polyhedral, rigid substrate described by the disclosure is configured to fit in a microcentrifuge tube and form fewer (e.g., 1, 2, 3, 4, 5 or more, fewer) constriction points in the tube relative to a circular substrate (e.g., a substrate that contacts substantially all of the inner surface of a microcentrifuge tube).

In some embodiments, a polyhedral, rigid substrate comprises an outer diameter that is smaller than the widest inner diameter of a microcentrifuge tube. An outer diameter refers to the longest distance between points (e.g., vertices) of the star polyhedron. In some embodiments, a polyhedral, rigid substrate comprises an outer diameter between about 5 mm and about 7 mm in length (e.g., 5.0 mm, 5.2 mm, 5.5 mm, 5.8 mm, 6.0 mm, 6.3 mm, 6.6 mm, 7.0 mm, or any length therebetween). In some embodiments, a polyhedral, rigid substrate comprises an outer diameter of 6 mm in length.

The thickness (e.g., height) of a polyhedral substrate may vary. In some embodiments, a polyhedral, rigid substrate comprises a thickness between 0.1 mm and 0.5 mm. In some embodiments, a polyhedral, rigid substrate comprises a thickness of 0.1 mm, 0.2 mm, 0.3 mm, 0.4 mm, or 0.5 mm.

The material used to form a polyhedral, rigid substrate may vary. Examples of materials include metals, certain polymers (e.g., plastics), silicate (e.g., borosilicate glass), etc. In some embodiments, a polyhedral, rigid substrate does not comprise cellulose-based paper (e.g., filter paper). In some embodiments, a polyhedral, rigid substrate comprises or consists of metal. In some embodiments, a metal comprises or consists of stainless steel. Examples of stainless steel include but are not limited to 301, 302, 303, 304, 309, 316, 321, 408, 409, 410, 416, 420, 430, 440, and 630 stainless steel. In some embodiments, the stainless steel is 304 stainless steel, 316 stainless steel, 420 stainless steel, or 440 stainless steel. Methods of forming metal substrates are generally known, and include for example punching, roll forming, extrusion, and press braking.

The polyhedral, rigid substrates described by the disclosure are typically substantially smooth. The term “substantially smooth” refers to a substrate having an even and regular surface or consistency that is free from perceptible projections, lumps, indentations, burrs or sharp edges. In some embodiments, each edge of a polyhedral, rigid substrate is substantially smooth. In some embodiments, each face of a polyhedral, rigid substrate is substantially smooth.

The disclosure is based, in part, on polyhedral substrates that are rigid. A rigid substrate typically refers to a substrate that does not deform when subjected to mechanical stress or force. In some embodiments, a polyhedral substrate has a rigidity that is higher than the rigidity of previously used DNA extraction substrates, for example borosilicate glass or cellulose-based filter paper. Rigidity may be measured in any conventional way, for example using Shear Modulus. In some embodiments, a substrate has a Shear Modulus ranging from between 20 to about 80 GPa (e.g., about 20, 30, 40, 50, 60, 70, or 80 GPa).

### Biological Samples

Methods described by the disclosure may be used to extract and/or purify nucleic acids from any suitable sample. The sample may be a biological sample, for example a fluid sample or a tissue sample. The sample is preferably a fluid sample. The sample typically comprises a body fluid. The body fluid may be obtained from a human or animal. The human or animal may have, be suspected of having or be at risk of a disease. The sample may be urine, lymph, saliva, mucus, seminal fluid or amniotic fluid, but is preferably whole blood, plasma or serum. Typically, the sample is human in origin, but alternatively it may be from another mammal such as from commercially farmed animals such as horses, cattle, sheep or pigs or may alternatively be pets such as cats or dogs.

Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton, tea or coffee.

The sample may be a non-biological sample. The non-biological sample is preferably a fluid sample. Examples of non-biological samples include surgical fluids, water such as drinking water, sea water or river water, and reagents for laboratory tests.

The sample may be processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

### Nucleic Acids

The disclosure relates, in some aspects, to nucleic acids and nucleic acid sequences. A “nucleic acid” sequence refers to a DNA or RNA (or a sequence encoded by DNA or RNA). In some embodiments, a nucleic acid is isolated. As used herein, with respect to nucleic acids, the term “isolated” means separated from other non-nucleic acid components (such as proteins, organelles, cellular debris, salts, buffers, etc.) as by mechanical or chemical separation, cleavage, gel separation, or any other suitable method. In some embodiments, DNA (e.g., HMW gDNA) is separated from proteins, RNA, and other cellular components using methods described herein. An isolated nucleic acid may be substantially purified. For example, a nucleic acid that is isolated is substantially pure even though it may comprise a tiny percentage of the material in the cell in which it resides.

In some embodiments, a nucleic acid or isolated nucleic acid is a referred to as a “polynucleotide” or “oligonucleotide”. The terms “polynucleotide” and “oligonucleotide” refer to nucleic acids comprising two or more units (e.g., nucleotides) connected by a phosphate-based backbone (e.g., a sugar-phosphate backbone), for example genomic DNA (gDNA), complementary DNA (cDNA), RNA (e.g., mRNA, shRNA, dsRNA, miRNA, tRNA, etc.), synthetic nucleic acids and synthetic nucleic acid analogs. Polynucleotides (or oligonucleotides) may include natural or non-natural bases, or combinations thereof and natural or non-natural backbone linkages, such as phosphorothioate linkages, peptide nucleic acids (PNA), 2′-O-methyl-RNA, or combinations thereof.

Aspects of the disclosure relate to methods for extracting and/or purifying large DNA molecules from biological samples. In some embodiments, a biological sample comprises high molecular weight (HMW) DNA. In some embodiments, HMW DNA comprises DNA of at least 30 kb in size. In some embodiments, HMW DNA comprises DNA of at least 50 kb, DNA of at least 100 kb, DNA of at least 200 kb, DNA of at least 300 kb, DNA of at least 500 kb, or DNA of at least 1 Mb in size. In some embodiments, DNA having a size greater than 50 kb is referred to as ultra-high molecular weight (UHMW) DNA.

After extraction, the genomic DNA (e.g., isolated genomic DNA) may be fragmented. The DNA may be fragmented by any suitable method. For example, methods of fragmenting DNA are known in the art. Such methods may use a transposase, such as a MuA transposase or a commercially available G-tube.

A nucleic acid (e.g., DNA) may be single stranded or double stranded. In some embodiments, a single stranded polynucleotide comprises a sequence of polynucleotides connected by a contiguous backbone. In some embodiments, a single stranded polynucleotide comprises a 5′ portion (end or terminus) and a 3′ portion (end or terminus). A single stranded polynucleotide may be a sense strand or an antisense strand.

In some embodiments, a nucleic acid (e.g., polynucleotide) is double stranded. A double stranded polynucleotide comprises a first (e.g., “sense”) polynucleotide strand that is hybridized to a second polynucleotide (“antisense”) strand via hydrogen bonding between the nucleobases of each strand along a region of complementarity between the two strands. Each strand of a double stranded polynucleotide comprises a 5′ potion and a 3′ portion.

### Nucleic Acid Extraction and Purification

Aspects of the disclosure relate to methods for isolating nucleic acids (e.g., DNA, such as gDNA or HMW DNA) from a biological sample, the method comprising: obtaining a biological sample comprising nucleic acids; contacting the biological sample with a polyhedral, rigid substrate; adsorbing the nucleic acids of the sample to the polyhedral, rigid substrate; washing the polyhedral, rigid substrate to remove non-nucleic acid components of the biological sample; releasing the nucleic acids from the polyhedral, rigid substrate to produce isolated nucleic acids from the biological sample.

Methods for extracting DNA from biological samples are known, and reagents and kits for doing so are commercially available. In some embodiments, DNA is extracted from a biological sample using a kit suitable for long-read or ultra-long read DNA sequencing. Examples of kits used for DNA extraction for long-read or ultra-long read DNA sequencing include but are not limited to Monarch® HMW DNA Extraction Kit (New England Biolabs, USA; neb.com/products/t3060-monarch-hmw-dna-extraction-kit-for-tissue #Product %20Information; accessed May 17, 2022), Wizard® HMW DNA Extraction Kit (Promega, WI, USA), QIAGEN MagAttract HMW DNA Kit, etc. Additional examples of HMW DNA extraction techniques are described, for example in US Patent Application Publication No. US 2021-0054363 A1, the entire contents of which are incorporated herein by reference.

In some embodiments, extracting DNA and/or RNA comprises lysing cells of a biological sample and isolating DNA and/or RNA from other cellular components. Examples of methods for lysing cells include, but are not limited to, mechanical lysis, liquid homogenization, sonication, freeze-thaw, chemical lysis, alkaline lysis, and manual grinding.

Methods for extracting DNA include, but are not limited to, solution phase extraction methods and solid-phase extraction methods. In some embodiments, a solution phase extraction method comprises an organic extraction method, e.g., a phenol chloroform extraction method. In some embodiments, a solution phase extraction method comprises a high salt concentration extraction method, e.g., guanidinium thiocyantate (GuTC) or guanidinium chloride (GuCl) extraction method. In some embodiments, a solution phase extraction method comprises an ethanol precipitation method. In some embodiments, a solution phase extraction method comprises an isopropanol precipitation method. In some embodiments, a solution phase extraction method comprises an ethidium bromide (EtBr)-Cesium Chloride (CsCl) gradient centrifugation method. In some embodiments, extracting DNA comprises a nonionic detergent extraction method, e.g., a cetyltrimethylammonium bromide (CTAB) extraction method.

In some embodiments, a solid-phase DNA extraction method comprises contacting a biological sample (e.g., a cell or tissue lysate comprising DNA) with a polyhedral, rigid substrate as described herein. In some embodiments, the contacting comprises providing conditions under which the nucleic acids of the sample nucleate on the surface of the substrate and form one or more nucleic acid aggregates. For example, addition of certain DNA precipitating agents (e.g., isopropanol, spermine, water, TE buffer, etc.) to the cell lysate causes the DNA in the sample to precipitate and aggregate on the surface of the polyhedral, rigid substrate. Without wishing to be bound by any particular theory, the polyhedral, rigid substrate provides a nucleation point for the precipitated DNA to form aggregates. The DNA aggregates may interact with the polyhedral, rigid substrate via any suitable mechanism, for example charge-interactions between DNA and the substrate surface molecules, adsorption, chelation, etc.

Such aggregates can then be washed using any suitable wash buffer and wash protocol. Wash buffers typically include alcohols (e.g., ethanol) and aid in removing salts and other ions from the biological sample, while leaving the precipitated DNA aggregated on the surface of the substrate. After addition of wash buffer, the sample may be centrifuged to collect undesirable components (e.g., proteins, salts, etc.) which may be removed from the sample. The sample may then be resuspended in wash buffer, and the process repeated one or more times (e.g., 1, 2, 3, 4, or more times). The inventors have recognized and appreciated that interaction between the polyhedral, rigid substrate and precipitated DNA reduces mechanical shear on the DNA during washing of the sample by reducing points of contact between the substrate and microcentrifuge walls.

After the final volume of wash buffer has been removed from the cells, the precipitated DNA may be eluted from the polyhedral, rigid substrate by contacting the DNA with any suitable elution buffer. Examples of suitable elution buffers include water (e.g., nuclease free water), Tris buffer, Tris-EDTA (TE) buffer, acetate buffers (e.g., ammonium acetate, etc.), or other low-ionic strength buffers. In some embodiments, the elution buffer comprises or consists of spermine. Spermine is typically a preferred elution agent when the isolated DNA will be used for additional library preparation techniques, for example techniques comprising use of certain enzymes, for example transposases or polynucleotide binding proteins. The eluted DNA may be referred to as isolated DNA.

Polyhedral, rigid substrates described by the disclosure may also be used to purify DNA that has been extracted by methods not including the use of polyhedral, rigid substrates (e.g., DNA that has previously been extracted using glass beads, or a solution-phase technique).

In some embodiments, a sample of extracted or purified DNA (e.g., HMW gDNA) is at least 100-20000 ng (e.g., 100-20000 ng, 500-15000 ng, 800-10000 ng, 1000-15000 ng, 1000-10000 ng, 1000-8000 ng, 1000-6000 ng, or 1000-2000 ng) in total mass. In some embodiments, a sample of extracted DNA is at least 1000-2000 ng in total mass. In some embodiments, the acceptable total DNA amount for further sequencing is at least 20-200 ng (e.g., 20-200 ng, 30-200 ng, or 50-150 ng). In some embodiments, the target total DNA (e.g., HMW gDNA) amount for further sequencing is more than 30-200 ng (e.g., 30-200 ng, 50-200 ng, or 100-200 ng).

In some embodiments, the target purity of a sample of extracted or purified DNA (e.g., HMW gDNA) is such that it corresponds to a range of a ratio of absorbance at 260 nm to absorbance at 280 nm of at least 1.8-2 (e.g., at least 1.8-2, at least 1.8-1.9). In some embodiments, the purity of a sample of extracted or purified DNA is such that it corresponds to a ratio of absorbance at 260 nm to absorbance at 280 nm of at least 1 (e.g., at least 1, at least 1.2, at least 1.4, at least 1.6, at least 1.8, or at least 2). In some embodiments, the acceptable purity of a sample of extracted or purified DNA is such that it corresponds to a ratio of absorbance at 260 nm to absorbance at 280 nm of at least 1.5 (e.g., at least 1.5, at least 1.7, at least 2). In some embodiments, the purity of a sample of extracted or purified DNA as described herein is analyzed by a spectrophotometer, for example a small volume full-spectrum, UV-visible spectrophotometer (e.g., Nanodrop spectrophotometer available from ThermoFisher Scientific).

Aspects of the disclosure relate to performing nucleic acid sequencing on isolated DNA (e.g., HMW gDNA) extracted and/or purified according to methods described herein. In some embodiments, one or more additional sample preparation techniques is performed on isolated DNA (e.g., HMW gDNA) produced according to methods described herein. For example, in some embodiments, isolated DNA is used as input for a library preparation method. Library preparation methods for sequencing are generally known, for example as described by Amarasinghe et al. Opportunities and challenges in long-read sequencing data analysis. Genome Biol 21, 30 (2020). doi.org/10.1186/s13059-020-1935-5. In some embodiments, a library preparation protocol comprises an Ultra-Long DNA Sequencing Kit protocol (e.g., as described by store.nanoporetech.com/us/ultra-long-dna-sequencing-kit.html, the entire contents of which are incorporated herein by reference).

Library preparation techniques typically include ligation of adaptors onto isolated nucleic acids (e.g., isolated HMW gDNA). In some embodiments, isolated DNA (e.g., HMW gDNA) templates are modified so that they comprise Y adaptors at both ends. Any manner of modification can be used. The method may comprise modifying the DNA (e.g., HMW gDNA) by adding the adaptors, such as Y adaptors, and/or anchors (e.g., tethers), by contacting the DNA (e.g., HMW gDNA) with a transposase enzyme (for example a MuA transposase) and a population of double stranded transposase substrates. The transposase fragments the DNA (e.g., HMW gDNA) and ligates the substrates to one or both ends of the fragments. This produces a plurality of modified DNA templates comprising an adaptor or anchor. The modified DNA templates may then be investigated. Example MuA-based methods are disclosed in WO 2015/022544 and WO 2016/059363, and WO2015/150786, the entire contents of each of which are incorporated herein by reference.

A sequencing adaptor may comprise a loading site for loading a polynucleotide binding protein. The loading site may be for instance a single-stranded region which can targeted by the polynucleotide binding protein.

The polynucleotide binding protein if present may be provided on a sequencing adaptor. WO 2015/110813 and WO 2020/234612, the entire contents of each of which are incorporated herein by reference, describe the loading of polynucleotide binding proteins onto a target polynucleotide such as an adaptor and are hereby incorporated by reference in their entireties.

### Nucleic Acid Sequencing

Aspects of the disclosure relate to methods of sequencing isolated nucleic acids (e.g., HMW gDNA) obtained from a biological sample from the subject. In some embodiments, the sequencing comprises a long-read or ultra-long read sequencing technique. However, the skilled artisan recognizes that isolated DNA extracted or purified according to methods described herein may also be used in short-read sequencing techniques.

The sequencing data may be obtained from the biological sample using any suitable sequencing technique and/or apparatus. In some embodiments, the sequencing apparatus used to sequence the biological sample may be selected from any suitable sequencing apparatus known including, but not limited to, Illumina, SOLid, Ion Torrent, PacBio, a nanopore-based sequencing apparatus (e.g., Oxford Nanopore Technologies PLC sequencing apparatus, such as MinION, GridION, PromethION), a Sanger sequencing apparatus, or a 454TM sequencing apparatus.

### Kits

Aspects of the disclosure relate to kits comprising polyhedral, rigid substrates as described herein. The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the system or kit in a dry state such that a fluid sample is used to resuspend the reagents. The system or kit may also, optionally, comprise instructions to enable the system or kit to be used in the methods described herein or details regarding for which organism the method may be used. The system or kit may comprise a magnet or an electromagnet. The system or kit may, optionally, comprise nucleotides. In some embodiments, the kit further comprises the components of an Oxford Nanopore Technologies PLC Ultra-Long DNA Sequencing Kit, including a lysis buffer, population of adapters, fragmentation mix (e.g., solution comprising transposase enzyme), and one or more buffers.

The following Examples illustrate the invention. It is to be understood that although particular embodiments, specific configurations as well as materials and/or molecules, have been discussed herein for methods according to the invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

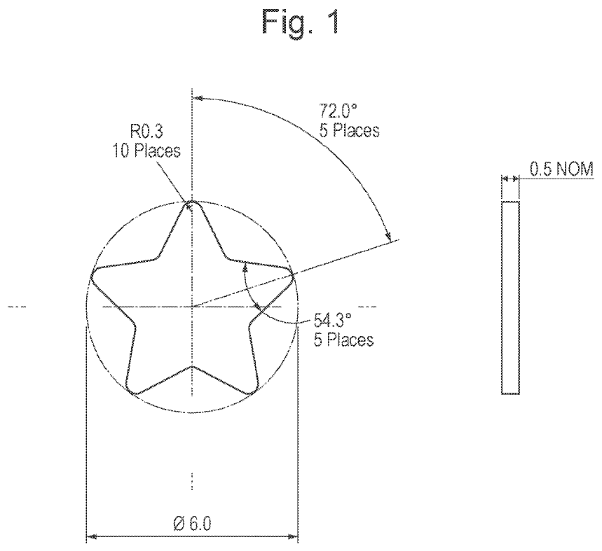

This example describes one embodiment of a polyhedral, rigid substrate. The substrate, shown in FIG. 1, comprises a regular star-shaped, stainless steel substrate that binds nucleic acids (e.g., DNA) present in biological samples.

The substrate has five points. The outer angle between each point is approximately 72 degrees, and the inner angle of each point is approximately 54.3 degrees. The substrate has a diameter (e.g., the distance between two opposing points of the star shape) of approximately 6 mm, and is approximately 0.5 mm thick. The substrate is a solid piece of 316 stainless steel, and each edge of the stainless steel is substantially smooth (e.g., free of burrs and sharp edges). In some embodiments, the surface of the substrate is acid-etched. Polyhedral substrates are advantageous over previously-described circular substrates because the polyhedral substrates do not form as many constriction points in the bottom of a microwell as circular substrates. Reduction in the number of constriction points reduces DNA shearing during DNA extraction, purification, and sequencing library preparation (or any other technique that involves spinning or centrifuging solubilized DNA).

The polyhedral substrate is rigid, meaning that the substrate is made of a material that does not flex or bend during manipulation with a pipette or during centrifugation (e.g., the substrate is less flexible than a shape made from cellulose filter paper). Rigid substrates do not spin all the way to the bottom of a sample preparation container (e.g., a microcentrifuge tube, such as an Eppendorf tube), and thus may allow for recovery of semi-eluted nucleic acids (e.g., DNA).

### Example 2

This example describes one embodiment of a protocol for extraction of genomic DNA (gDNA) using polyhedral, rigid substrates described by the disclosure. In a sample preparation tube (e.g., a 1.5 mL Eppendorf tube), a homogenate containing cells and/or tissue is lysed using surfactants, and proteins are denatured with guanidine HCl.

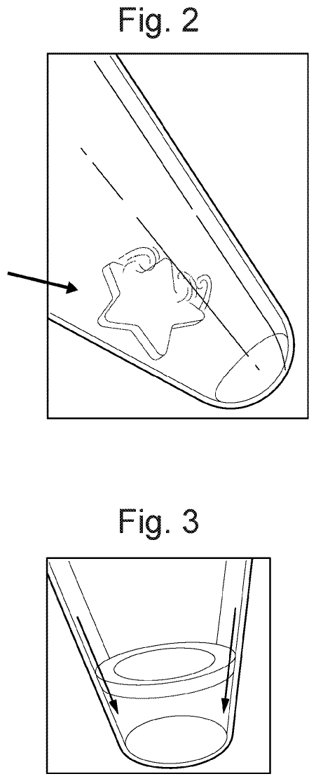

Next, a polyhedral-rigid substrate (e.g., 5 mm stainless steel stars) are added to the lysate. DNA (including gDNA) is then precipitated out of the solution using Isopropanol (ISOP). As the gDNA precipitates, it nucleates around the polyhedral rigid substrate (e.g., the metal star) and aggregates at the surface of the substrate (FIG. 2). The gDNA can then be washed while it interacts (e.g., binds or aggregates on) the substrate, or be eluted off the substrate into a buffer of choice, for example water, isopropanol, or spermine.

Subsequent additional sample preparation techniques, for example fragmentation, ligation of adaptors for sequencing, etc., may be performed on the eluted gDNA. When preparing a sample for nanopore-based sequencing, gDNA is preferably precipitated out of the solution using combination of spermine (or equivalents, such as other polyamines or polycations) and metal stars because ISOP may denature molecular motors or other proteins useful for downstream preparation of gDNA for sequencing.

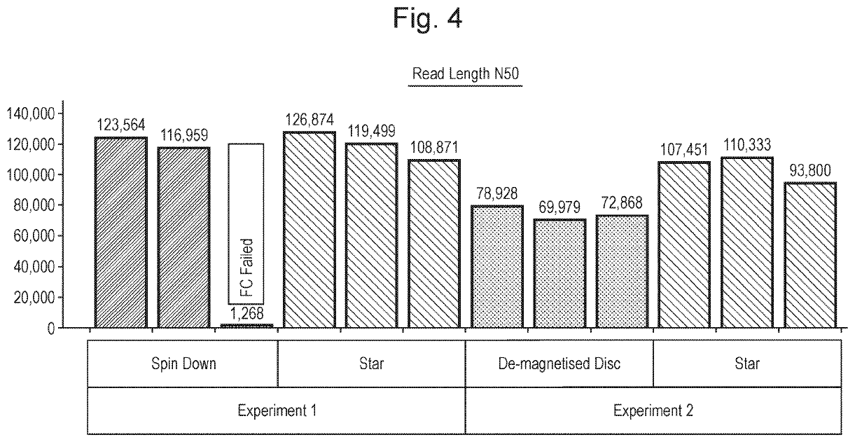

One advantage of using a non-circular substrate (e.g., a polyhedral, rigid substrate) for DNA extraction and purification is that polyhedral, rigid substrates, unlike circular solid substrates, do not form construction points through which DNA (e.g., gDNA) is forced during precipitation and washing steps, which often include centrifugation. These constriction points (FIG. 3, arrows) have been observed to contribute to shearing of DNA (including gDNA), resulting in shorter DNA fragments being isolated from the sample. The shorter fragments of DNA are one cause of reduced read lengths when performing nucleic acid sequencing. FIG. 4 shows representative data indicating read length N50s (Read N50 refers to a value where half of the data is contained within reads with lengths greater than this; thus N50 is the length of the sequence in a set for which all sequences of that length or greater sum to 50% of the set's total size) from sequencing runs where a DNA library was cleaned up using rigid, polyhedral substrates described herein (e.g., metal star shapes). Data indicate the star shaped substrate appears to protect the DNA from shearing whereas extraction by circular shaped substrates causes DNA shearing.

### Example 3

This example provides one embodiment of a workflow for preparation of high molecular weight (HMW) DNA from a biological sample for nanopore-based sequencing. In some embodiments, a wide-bore pipette tip is used for all pipetting and mixing steps. DNA extraction is performed using a Monarch® HMW DNA Extraction Kit (New England Biolabs, USA; neb.com/products/t3060-monarch-hmw-dna-extraction-kit-for-tissue #Product %20Information; accessed May 17, 2022). Purification of extracted gDNA and subsequent ligation of sequencing adaptors is performed using Ultra-Long DNA Sequencing Kit (Oxford Nanopore Technologies PLC, UK; store.nanoporetech.com/us/ultra-long-dna-sequencing-kit.html; accessed May 17, 2022) combined with a polyhedral, rigid substrate (e.g., metal star) as described by the disclosure.

### DNA Extraction:

1. Make up 1800 μL Monarch HMW gDNA Tissue Lysis Buffer+60 μL Proteinase K in 2 mL tube
2. Take 6e6 cells from cell culture (e.g., GM24385 (10e6/ml)=600 ul for 6e6).
3. Transfer the cells to a 5 mL tube.
4. Pellet by centrifuging at 500×g 3 min
5. Remove supernatant, attempt not to disturb cell pellet.
6. Re-suspend in 40 μL PBS, flick mix.
7. Add 1800 μL Lysis and Proteinase K buffer.
8. Mix 5× times with wide bore pipette.
9. Add 15 μL RNAase A. Mix 5× times with wide bore.
10. Heat 56° C. 10 minute shaking 650 rpm.
11. Add 900 μL of Monarch Protein Separation Solution without mixing. Mix using a hula mixer for 10 minutes at the lowest speed at which the movement of the mixer is smooth). This step is necessary for tissue extraction but not for all cell lines.
12. Centrifuge at 4° C. 16000 g for 10 minutes to separate the protein and DNA.
13. Aspirate DNA containing upper phase leaving behind lower phase which contains the protein, being careful not to take up any of lower phase and dispense into a new 5 mL tube.
14. Add 3× glass beads to the tube (one will remain at the bottom of the tube).
15. Add 2500 μL of isopropanol to the tube and mix on a hula mixer for approximately 20 minutes.
16. Remove isopropanol supernatant, making sure not to remove any DNA bound to the beads. Remove any isopropanol that might also be stuck in the lid.
17. Wash with 2 mL Monarch gDNA wash Buffer.
18. Remove wash buffer.
19. Wash with 2 mL Monarch gDNA wash Buffer.
20. Set up 2 mL tube with 560 μL EEB (e.g., TE pH 9.0, 0.02% EcoSurf).
21. Discard most of wash buffer and then transfer remaining and the glass beads to a spin column.
22. Spin down gently to remove residual Wash Buffer, using bench top centrifuge.
23. Transfer glass beads from bead retainers into tube containing EEB for elution.
24. Heat at 56° C. 10 minutes.
25. Remove beads from EEB by pouring into a bead retainer in a 2 mL tube and keep EEB, spin to remove EEB remaining on the beads.
26. Discard glass beads and retainer then add extra 200 μL of EEB to elute.
27. Incubate overnight at room temperature.

### Purification and Library Prep:

1. Combine 6 μL of FRA with 244 μL of FDB and mix thoroughly.
2. Add 250 μL of FRA/FDB added to the 750 μL of gDNA and gently mix.
3. Incubate at room temperature for 10 min.
4. Incubate at 75° C. for 10 min to inactivate protein complexes (e.g., transposome complex).
5. Cool down on ice.
6. Add 5 μL of poly T RAP, Mix gently and thoroughly.
7. Incubate for 30 min at room temperature.
8. Add metal star.
9. Add 500 μL of PPT reagent. 20 mM SPERMINE.
10. Rotate at slow speed for 20 min on hula mixer.
11. Remove supernatant, leaving enough so that the metal star is still suspended.
12. Pour metal star and left-over supernatant into 1.5 mL tube.
13. Remove rest of supernatant from beneath the star.
14. Gently spin to remove residual supernatant in benchtop centrifuge.
15. Pipette out the residual supernatant from bottom of tube.
16. Add 225 μL of EB and leave to re-suspend overnight.

After extraction, purification, and library preparation, the nucleic acid sample is loaded into a nanopore sequencing flow cell (e.g., FLO-MIN106D, FLO-MIN111, FLO-PRO002; Oxford Nanopore Technologies PLC, UK) for example according to the protocol below. Samples are then run on a sequencing apparatus, for example MinION, GridION, PromethION, etc.).

### Flow Cell Loading

1. Pipette out as much as possible of the EB from the tube containing the star into new 1.5 mL tube.
2. Spin down the tube on a benchtop centrifuge.
3. Remove all liquid from the tube and make sure there is no liquid left on the metal star.
4. Mix sample thoroughly with a wide bore pipette tip.
5. Take 75 μL of the sample into a new tube and add 75 μL of SQB, mix thoroughly with wide bore pipette.
6. Leave to incubate at room temperature for 30 minutes.
7. Prime R9.4.1 PromethION flowcells with priming mix (1170 μL of FB and 30 μL of Priming tether per flowcell).
8. Load full 150 μL library using a wide bore pipette.
9. Run on sequencing apparatus.

What is claimed is:

1. A method for isolating nucleic acids from a biological sample, the method comprising:
(a) obtaining a biological sample comprising nucleic acids;
(b) contacting the biological sample with a polyhedral, rigid substrate;
(c) adsorbing the nucleic acids of the sample to the polyhedral, rigid substrate;
(d) washing the polyhedral, rigid substrate to remove non-nucleic acid components of the biological sample;
(e) releasing the nucleic acids from the polyhedral, rigid substrate to produce isolated nucleic acids from the biological sample.

2. The method of claim 1, wherein the nucleic acids comprise DNA, optionally wherein the DNA is genomic DNA (gDNA).

3. The method of claim 1 or 2, wherein the nucleic acids comprise high molecular weight (HMW) DNA.

4. The method of claim 3, wherein the HMW DNA comprises at least 30 kb.

5. The method of any one of claims 1 to 4, wherein the HMW DNA comprises DNA of at least 50 kb, DNA of at least 100 kb, DNA of at least 200 kb, DNA of at least 300 kb, DNA of at least 500 kb, or DNA of at least 1 Mb in size.

6. The method of any one of claims 1 to 5, wherein the biological sample comprises a tissue sample, blood sample, tissue lysate, or cell lysate.

7. The method of claim 6, wherein the tissue lysate or cell lysate has been previously obtained from the biological sample.

8. The method of any one of claims 1 to 7, wherein obtaining the biological sample comprises lysing cells or tissue of the biological sample to produce a cell lysate or tissue lysate.

9. The method of any one of claims 1 to 9, wherein the polyhedral, rigid substrate comprises between 4 and 50 faces.

10. The method of any one of claims 1 to 9, wherein the polyhedral, rigid substrate comprises 12 faces.

11. The method of any one of claims 1 to 10, wherein the polyhedral, rigid substrate forms a regular shape.

12. The method of any one of claims 1 to 11, wherein the polyhedral, rigid substrate forms an irregular shape.

13. The method of any one of claims 1 to 12, wherein the polyhedral, rigid substrate forms a star shape.

14. The method of any one of claims 1 to 11, wherein the star shape is a regular star shape.

15. The method of claim 13 or 14, wherein the star shape comprises five points.

16. The method of any one of claims 13 to 15, wherein each outer angle of the star shape is between 71 and 73 degrees.

17. The method of any one of claims 13 to 16, wherein each inner angle of each point of the star shape is between 53 and 56 degrees.

18. The method of any one of claims 1 to 17, wherein each edge of the polyhedral, rigid substrate is substantially smooth.

19. The method of any one of claims 1 to 18, wherein the polyhedral, rigid substrate comprises an outer diameter between about 5 mm and about 7 mm in length

20. The method of any one of claims 1 to 19, wherein the polyhedral, rigid substrate comprises an outer diameter of 6 mm in length.

21. The method of any one of claims 1 to 20, wherein the polyhedral, rigid substrate comprises a thickness between 0.1 mm and 0.5 mm.

22. The method of any one of claims 1 to 21, wherein the polyhedral, rigid substrate comprises or consists of metal.

23. The method of claim 22, wherein the metal comprises stainless steel, optionally wherein the stainless steel is 304 stainless steel, 316 stainless steel, 420 stainless steel, or 440 stainless steel.

24. The method of any one of claims 1 to 23, wherein adsorbing the nucleic acids of the sample to the polyhedral, rigid substrate comprises providing conditions under which the nucleic acids of the sample nucleate on the surface of the substrate and form one or more nucleic acid aggregates.

25. The method of claim 24, wherein the conditions comprise contacting the biological sample with isopropanol, spermine, water, or TE buffer.

26. The method of any one of claims 1 to 25, wherein washing comprises centrifuging the biological sample after contacting the biological sample with a wash buffer.

27. The method of any one of claims 1 to 26, wherein the non-nucleic acid components removed from the biological sample comprise one or more proteins, RNA molecules, salts, carbohydrates, or other cellular debris.

28. The method of any one of claims 1 to 27, wherein releasing the nucleic acids from the substrate comprises contacting the substrate with isopropanol, spermine, water, or TE buffer.

29. The method of any one of claims 1 to 28, wherein the isolated nucleic acids comprise or consist of genomic DNA (gDNA).

30. The method of any one of claims 1 to 29, wherein the isolated nucleic acids comprise or consist of high molecular weight (HMW) DNA, optionally wherein the HMW DNA comprises DNA of at least 30 kb in size.
